(19) United States
(12) Patent Application Publication
(10) Pub. No.: US 2026/0126057 A1
(43) Pub. Date: May 7, 2026

(54) PERSONAL TEMPERATURE CONTROL DEVICE

(71) Applicant: BLUHALO LLC, Chicago, IL (US)

(72) Inventor: Gareth Ambrose Woods, Chicago, IL (US)

(21) Appl. No.: 18/939,686

(22) Filed: Nov. 7, 2024

(51) Int. Cl. F04D 29/58; A61F 7/00; F25B 21/02

(52) U.S. Cl. CPC F04D 29/5826; F25B 21/02; A61F 2007/0064

(57) ABSTRACT

A personal temperature control device is provided. In some aspects, the personal temperature control device includes a housing having a body with a bottom surface, a top surface, a front surface, and a rear surface defining a body interior, a fan that provides air flow, wherein the fan is arranged in the body interior, and a power source that powers the fan. The personal temperature control device also includes a curved conduit that extends from an opening in the bottom surface and along the rear surface of the body, the curved conduit comprising a main channel connected to an output of the fan, wherein the curved conduit directs air flow from the fan and secures the personal temperature control device to a wearable item on a person.

### TECHNICAL FIELD

This disclosure relates generally to portable electronic devices, and more particularly, to a personal temperature control device.

### BACKGROUND

Activities performed by law enforcement officers, construction workers, military personnel, firefighters, plant workers, chefs, security personnel, and others, often necessitate specialized clothing, vests, body armor, and so forth. However, such protective items tend to be heavy and bulky, and are often made from materials of limited breathability. Insufficient ventilation from heavy, bulky, and/or non-breathable items can cause significant discomfort to personnel and workers, as well as raise risk for overheating or heat stroke.

Thus, a need exists improved technologies that can achieve personal cooling.

### SUMMARY

In accordance with one aspect of the present disclosure, a personal temperature control device is provided. In one embodiment, the personal temperature control device includes a housing having a body with a bottom surface, a top surface, a front surface, and a rear surface defining a body interior, a fan that provides air flow, wherein the fan is arranged in the body interior, and a power source that powers the fan. Further, in some embodiments, the personal temperature control device also includes a curved conduit that extends from an opening in the bottom surface and along the rear surface of the body, the curved conduit comprising a main channel connected to an output of the fan. The curved conduit is configured to direct air flow from the fan and secure the personal temperature control device to a wearable item on a person.

In accordance with another aspect of the present disclosure, a personal temperature control device is provided. In one embodiment, the personal temperature control device includes a housing having a body with a bottom surface, a top surface, a front surface, and a rear surface defining a body interior, and a fan that provides air flow, wherein the fan is arranged in the body interior. Further, in some embodiments, the personal temperature control device also includes a curved conduit that extends from an opening in the bottom surface and along the rear surface of the body, the curved conduit comprising a main channel connected to an output of the fan, and a spring clip attached to the rear surface of the body. The curved conduit is configured to engage with the spring clip to secure the personal temperature control device to a wearable item on a person, and direct air flow under the wearable item.

In accordance with yet another aspect of the present disclosure, a method for manufacturing a personal temperature control device is provided. In some implementations, the method includes forming a housing having a body with a bottom surface, a top surface, a front surface, and a rear surface defining a body interior, and a curved conduit extending from the bottom surface along the rear surface of the body. The method also includes arranging, in the body interior, a fan that provides air flow to a main channel in the curved conduit, arranging, in the body interior, a power source that selectively powers the fan, wherein the curved conduit is configured to direct air flow from the fan and secure the personal temperature control device to a wearable item on a person.

The foregoing and additional aspects and implementations of the present disclosure will be apparent to those of ordinary skill in the art in view of the detailed description of various embodiments and/or implementations, which is made with reference to the drawings, a brief description of which is provided next.

### BRIEF DESCRIPTION OF THE DRAWINGS

The foregoing and other advantages of the present disclosure will become apparent upon reading the following detailed description and upon reference to the drawings.

FIG. 1 is a schematic diagram of an example personal temperature control device, according to some embodiments of the present disclosure.

FIG. 2A is a perspective view of an example of the personal temperature control device of FIG. 1, according to some embodiments of the present disclosure.

FIG. 2B is a front view of the personal temperature control device of FIG. 2A, according to some embodiments of the present disclosure.

FIG. 2C is a rear view of the personal temperature control device of FIG. 2A, according to some embodiments of the present disclosure.

FIG. 2D is a first side view of the personal temperature control device of FIG. 2A, according to some embodiments of the present disclosure.

FIG. 2E is a second side view of the personal temperature control device of FIG. 2A, according to some embodiments of the present disclosure.

FIG. 2F is a bottom view of the personal temperature control device of FIG. 2A, according to some embodiments of the present disclosure.

FIG. 2G is a top view of the personal temperature control device of FIG. 2A, according to some embodiments of the present disclosure.

FIG. 2H is a perspective view of the personal temperature control device of FIG. 2H with a portion of the housing removed, according to some embodiments of the present disclosure.

FIG. 3A is a front view of the personal temperature control device of FIG. 2A with a lanyard attached thereto, according to some embodiments of the present disclosure.

FIG. 3B is a perspective view of the personal temperature control device of FIG. 2A with a lanyard attached thereto, according to some embodiments of the present disclosure.

FIG. 4 is a flowchart setting forth example steps of a process, according to aspects of the present disclosure.

Certain implementations have been shown by way of example in the drawings and will be described in further detail herein. It should be understood, however, that the present disclosure is not intended to be limited to particular forms disclosed. Rather, the present disclosure covers all modifications, equivalents, and alternatives falling within the spirit and scope of the present disclosure as defined by the claims.

### DETAILED DESCRIPTION

Law enforcement officers, construction workers, military personnel, firefighters, plant workers, and others, typically wear specialized clothing, vests, body armor, and so forth. While conventional hand-held or stationary ventilation devices can be useful in cooling, such devices can be difficult and/or ineffective with activities and/or attire worn by such personnel and workers. For example, many activities performed by such personnel require the use of the person's hand and/or prohibit the person from being stationary near such stationary ventilation devices.

The systems, methods, and apparatus disclosed herein seek to minimize, if not eliminate, the problems with current systems. In one embodiment, a personal temperature control device is provided. The personal temperature control device can be securely attached to a wearable item, such as a protective vest, body armor, and so forth. The personal temperature control device is configured to provide controllable air flow under such wearable items.

The present disclosure is described with reference to the attached figures, where like reference numerals are used throughout the figures to designate similar or equivalent elements. The figures are not drawn to scale, and are provided merely to illustrate the instant disclosure. Several aspects of the disclosure are described below with reference to example applications for illustration.

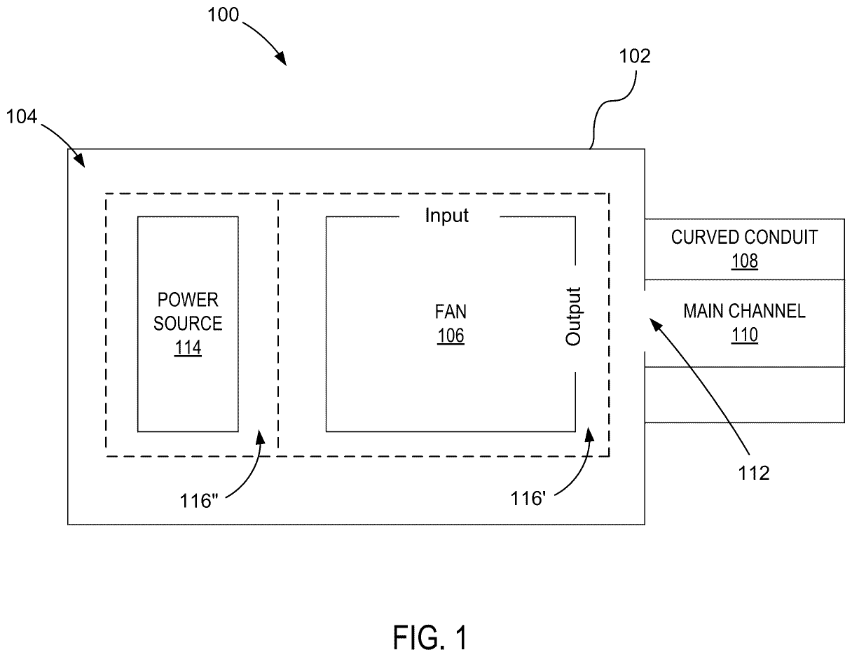

Referring to FIG. 1, an example of a personal temperature control device 100, in accordance with aspects of the present disclosure, is illustrated. In some embodiments, the personal temperature control device 100 may include a housing 102 having a body with various surfaces (e.g., a bottom surface, a top surface, a front surface, a rear surface, and other surfaces) that define a body interior 104. While the present disclosure uses terms like “top,” “bottom,” “front,” and “rear” with regard to surfaces shown and described, the orientation and perspective of such surfaces is arbitrary, and hence may vary.

The housing 102 of the personal temperature control device 100 may have various features and may be formed using various components and materials. For example, the housing 102 may be formed using a durable or rigid material, such as a plastic, metal, rubber, and so forth. In some embodiments, the housing 102 may include a first portion and a second portion, which may be removably coupled, for example, using any combination of fasteners, tabs, and so forth. In this manner, the housing 102 may be opened and closed to provide access to various components therein.

The body interior 104 may have various features and may be configured or designed to hold and/or secure a variety of components therein. For instance, in some embodiments, the personal temperature control device 100 may include a fan 106 arranged in the body interior 104, where the fan 106 may be selectively operated to generate air flow between an input and an output of the fan 106. In some embodiments, the input to the fan 106 may be near, or connected to an opening, grill, mesh, slot, and so forth, formed in the housing 102, which supplies the input of the fan 106 with air. In some non-limiting examples, the fan 106 may be an axial fan, a centrifugal fan, a blower fan, a bladeless fan, and so forth.

Extending from the housing 102 is a curved conduit 108 having a main channel 110 connected to the output of the fan 106 via an opening 112 in the housing 102. The curved conduit 108 may include any number, combinations, and arrangements of linear portions (e.g., straight) and non-linear portions (e.g., curved, angled, and so forth). The main channel 110 extends from a channel inlet to a channel outlet, where the channel inlet is proximal to the opening 112 in the housing 102. In some embodiments, the curved conduit 108 is configured to (e.g., by way of shape, dimension, material, and so forth) to direct air flow generated by the fan 106. For instance, the curved conduit 108 can provide cooling and/or heating to a person by virtue of controlled or controllable air flow through the main channel 110 in the curved conduit 108. In particular, the main channel 110 of the curved conduit 108 may be shaped and/or dimension, for instance, to control flow or flow impedance therethrough, as well as other aspects. In this manner, more efficient or effective temperature control of a person wearing the wearable item may be achieved.

As described further herein, in some embodiments, the curved conduit 108 may also be configured to secure the personal temperature control device 100 to a wearable item (e.g., clothing, vest, body armor, and so forth) on the person. For instance, at least a portion of the curved conduit 108 may be inserted underneath the wearable item. To this end, the curved conduit 108 may be shaped and/or dimensioned to facilitate receiving and engaging the wearable item. For example, the curved conduit 108 may be shaped and/or dimensioned to allow and facilitate the personal temperature control device 100 to engage and secure to the wearable item. In some embodiments, the curved conduit 108 may be used in cooperation with other components and/or features (e.g., a spring clip) on the personal temperature control device 100 to secure the personal temperature control device 100 to the wearable item.

To power various components therein, the personal temperature control device 100 may include a power source 114. In some embodiments, the power source 114 may include one or more devices that may store and/or deliver electric power, such as one or more rechargeable battery, one or more removable battery, one or more solar cell, and so forth. Power provided by the power source 114 may be delivered using various components and hardware (e.g., circuitry, circuit board, wiring, and so forth), and may be selectively controlled using various components and hardware (e.g., button, switch, potentiometer, slider, microcontroller, and so forth) arranged in or about the personal temperature control device 100.

The personal temperature control device 100 may also include other components and features for additional use, convenience, and/or functionality of the personal temperature control device 100. For instance, in some embodiments, the personal temperature control device 100 may include at least one source of light (e.g., LED, light bulb, and so forth), which may be selectively powered by the power source 114. Also, in some embodiments, the personal temperature control device 100 may include a charging port for charging the power source 114.

By virtue of components operating inside the personal temperature control device 100 (e.g., the fan 106, a light source, and so forth), the personal temperature control device 100 and/or air flow provided may get warm with time. To this end, the personal temperature control device 100 may implement any number of ways to control temperature of the personal temperature control device 100, components therein, and air flow provided by the personal temperature control device 100. For example, the personal temperature control device 100 may implement a passive cooling approach, for example, via one or more opening, slot, or vent in the housing 102 allowing heat to escape from the body interior 104, and/or a thermal mass (e.g., a heat sink) arranged therein. The personal temperature control device 100 may alternatively, or additionally, implement an active cooling approach. For instance, in some embodiments, the personal temperature control device 100 may include a Peltier cell for controlling a temperature of the personal temperature control device 100, and/or various components therein, as well as air flow provided. For instance, a first side of the Peltier cell may be arranged to face a body exterior, and a second side of the Peltier cell may be arranged to face the body interior 104. To provide efficient cooling, in some embodiments, a heat sink may be attached to the first side of the Peltier cell. A conductive mass may also be attached to the second side of the Peltier cell. In some embodiments, at least a portion of air flow provided by the fan 106 may be exposed to the conductive mass (or component thermally linked thereto), thereby providing a cooling air flow into the main channel 110. For instance, at least a portion of a flow conduit connecting the output of the fan 106 and the channel inlet of the main channel 110 (not shown in FIG. 1) may be thermally linked to the conductive mass.

As described, the housing 102 and/or body interior 104 of the personal temperature control device 100 may include various features. For instance, the body interior 104 may optionally include one or more compartments 116 or recesses formed in the body interior 104 that may hold and/or secure various components therein. For example, as illustrated in FIG. 1, the body interior 104 may include a first compartment 116′ that may hold and/or secure the fan 106, and a second compartment 116″ that may hold and/or secure the power source 114. In some embodiments, the compartment(s) 116 may be formed using one or more walls or dividers extending from one or more surfaces of the housing 102. Such wall(s) or divider(s), as well as other features may be shaped and/or dimensioned to hold and/or secure various components therein, as well as provide access to such components. For instance, one or more wall or divider forming the first compartment 116′ holding the fan 106 may have at least one opening to allow air flow to the input of the fan 106, to allow air flow from the output of the fan 106, to provide an electrical connection powering the fan 106, and so forth.

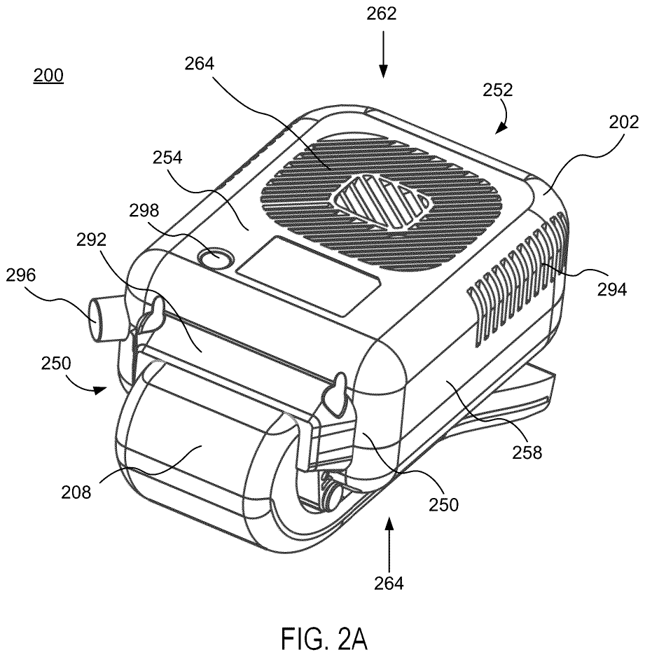

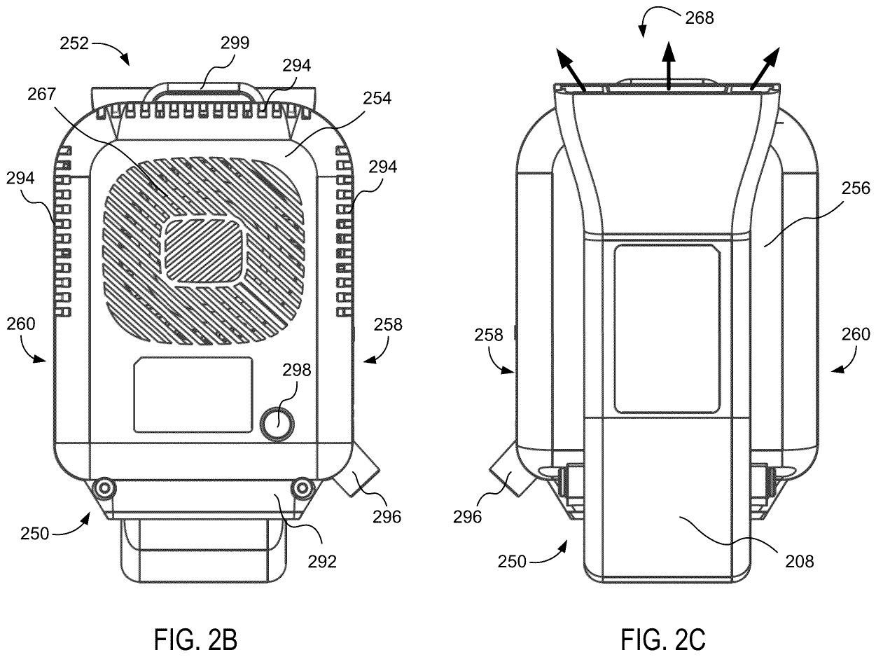

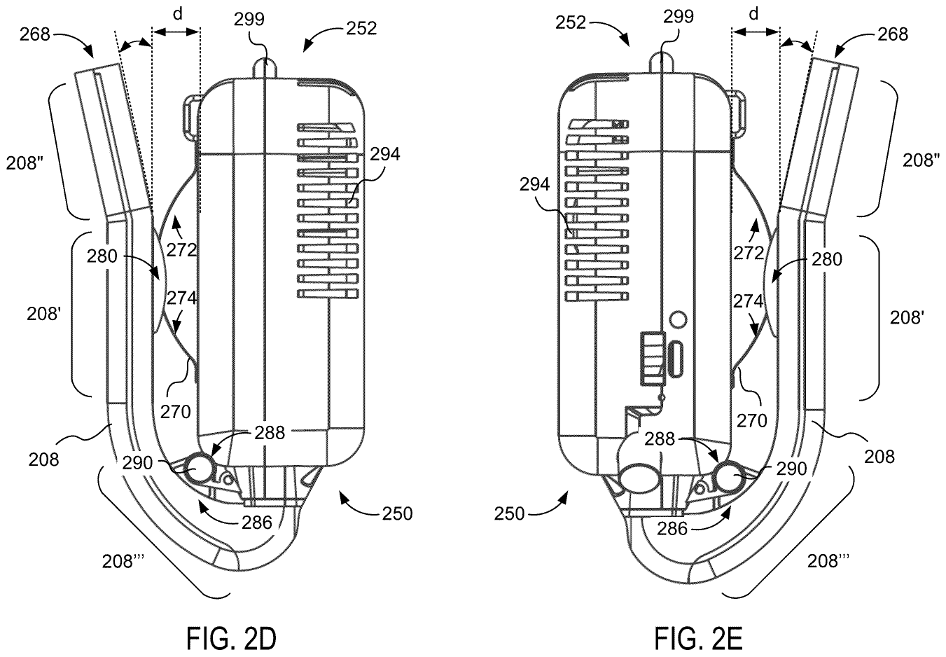

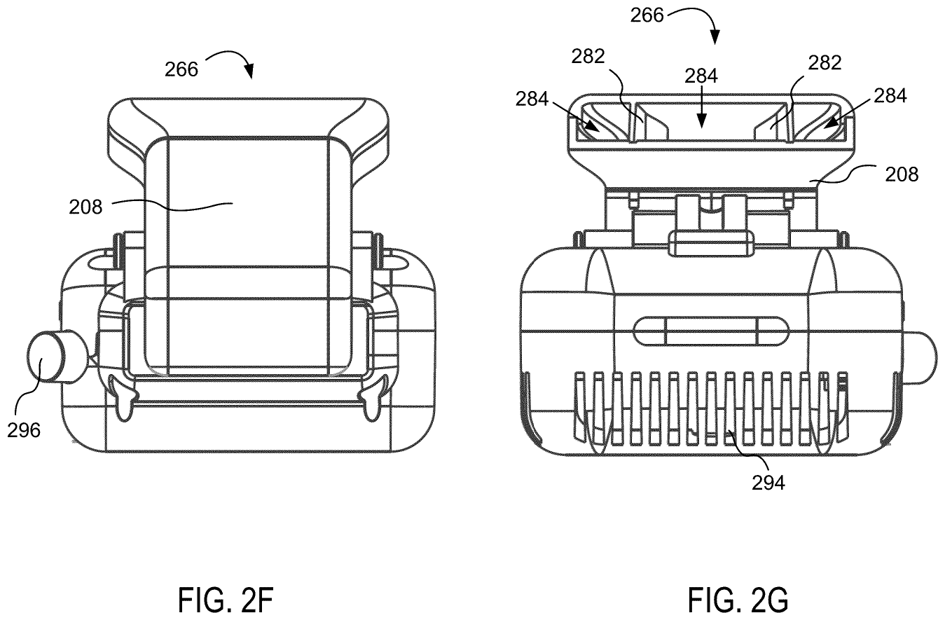

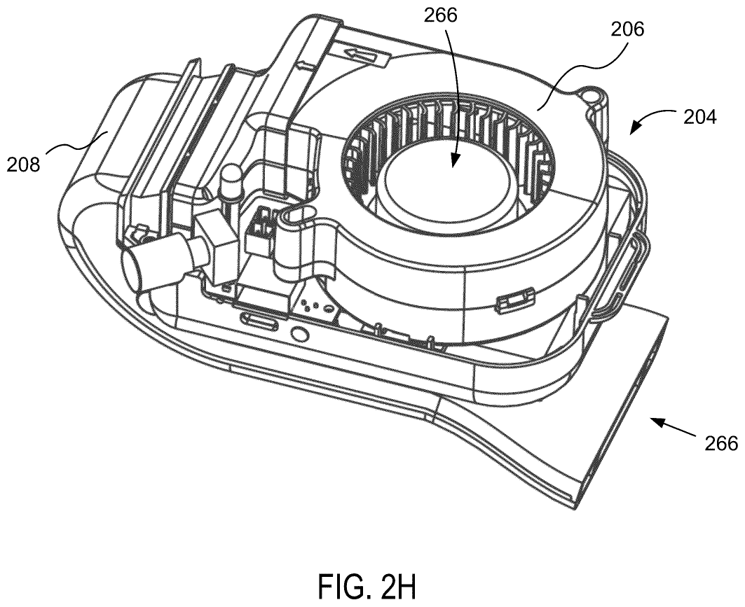

Turning now to FIGS. 2A-2H, one example a personal temperature control device 200, in accordance with aspects of the present disclosure, is illustrated. As shown, in some embodiments, the personal temperature control device 200 may include a housing 202 having a body with at least a bottom surface 250, a top surface 252, a front surface 254, a rear surface 256, a first side surface 258, and a second side surface 260 that define a body interior 204. The housing 202 may have various features and may be formed using various components and materials. For example, the housing 202 may be formed using a durable material or rigid material, such as a plastic, metal, rubber, and so forth. In some embodiments, the housing 202 may include a first portion 262 and a second portion 264 (e.g., FIG. 2A), which may be removably coupled, for example, using any combination of fasteners, tabs, clips, and so forth. In this manner, the housing 202 may be movably opened and closed to provide access to various components therein (e.g., FIG. 2H).

As illustrated in FIG. 2H, the body interior 204 may include various features, and may be configured to hold and/or secure a variety of components therein. For instance, in some embodiments, the personal temperature control device 200 may include a fan 206 arranged in the body interior 204, where the fan 206 may be selectively operated to generate air flow between a fan input 266 and a fan output. In some embodiments, the fan input 266 may be near, or connected to, a grill 267 formed in the housing 202, where in the grill 267 includes a number of small openings that allow exterior air into the housing 202 and the fan input 266.

Extending from the housing 202 is a curved conduit 208 having a main channel 210 connected to the output of the fan 206 via an opening in the housing 202. The main channel 210 extends from a channel inlet to a channel outlet 268, where the channel inlet is proximal to the opening in the housing 202. In some embodiments, the curved conduit 208 may be shaped, dimensioned, and/or formed to direct controlled air flow generated by the fan 206, for instance, to provide cooling and/or heating to a person by virtue of air flow through the main channel 210 in the curved conduit 208.

As described, in some embodiments, the curved conduit 208 may also be configured to secure the personal temperature control device 200 to a wearable item (e.g., clothing, vest, body armor, and so forth) on the person. To this end, the curved conduit 208 may be shaped, dimensioned, and/or formed to facilitate receiving and engaging the wearable item. For instance, in some embodiments, the curved conduit 208 may include a first conduit portion 208′ that is displaced relative to a plane defined by a rear surface 256 of the body of the housing 202. For example, as shown in FIGS. 2D-2E, the first conduit portion 208′ may be displaced by a distance d, which may vary between approximately 5 mm and 5 cm, although other values for d may be possible.

In some embodiments, the curved conduit 208 may also include a second conduit portion 208″ that is angled relative to the plane defined by the rear surface 256 of the body of the housing 102. For example, as shown in FIGS. 2D-2E, the second conduit portion 208″ may form an angle δ relative to a vertical axis defined by the plane of the rear surface 256, where the angle δ may vary between approximately 5 degrees and approximately 20 degrees, although other values for δ may be possible. The displaced, first conduit portion 208′ and angled, second conduit portion 208″ form a space between the curved conduit 208 and the housing 202, which allows and facilitates the personal temperature control device 200 to engage and secure to the wearable item in the space.

As illustrated in FIGS. 2D-2E, in some embodiments, the curved conduit 208 may also include a third conduit portion 208′″, which includes a curvature that allows the curved conduit 208 to re-direct air flow provided by the fan 206. In the example of FIGS. 2A-2H, air flow provided by the fan 206 flows in a direction from a top surface 252 to a bottom surface 250, and exits the body of the housing 202 through an opening in the bottom surface 250. By virtue of the shape of the curved conduit 208, and particularly the curvature in the third conduit portion 208′″, airflow exiting from the bottom surface 250 is re-directed upward toward the top of the personal temperature control device 200.

As described, in some embodiments, the personal temperature control device 200 may include a spring clip 270 attached to the rear surface 256 of the body of the housing 202 (e.g., FIGS. 2D-2E). The spring clip 270 may engage with the curved conduit 208 to secure the personal temperature control device 200 to the wearable item. The spring clip 270 may have any shape and dimension. For instance, the spring clip 270 may be configured with sufficient rigidity to provide a force that pushes back against the wearable item and holds a weight of the personal temperature control device 200 when the curved conduit 208 and the spring clip 270 engage the wearable item. The spring clip 270 may also be configured with sufficient elasticity to flex, as well as revert to its original shape when the curved conduit 208 and the spring clip 270 disengage the wearable item. In some embodiments, the spring clip 270 may include a shim or strip of material (e.g., a metal, plastic, and so forth). For example, the spring clip 270 may include a flexible shim or flexible strip made using steel.

For instance, as illustrated in FIGS. 2D-2E, in some embodiments, the spring clip 270 may extend along an axis defined by the top surface 252 and the bottom surface 250 of the body of the housing 202, where the spring clip 270 includes a first curvature 272 and a second curvature 274 along the axis. In particular, the first curvature 272 of the spring clip 270 may facilitate insertion of a portion of the wearable item between the spring clip 270 and the curved conduit 208, and the second curvature 274 of the spring clip 270 may secure the portion of the wearable item between the spring clip 270 and the curved conduit 208. In some embodiments, the spring clip 270 may be received by a spring clip slot formed in the curved conduit 208. Referring again to FIGS. 2D-2E, in some embodiments, the spring clip slot may be formed in a raised portion 280 of the curved conduit 208.

As appreciated from description herein, the curved conduit 208 may be positioned to direct air flow underneath a wearable item. To this end, at least a portion of the curved conduit 208 may be inserted underneath the wearable item. In some embodiments, the curved conduit 208 may be rigid, for instance, by virtue of a rigid material used to form the curved conduit 208, and/or by virtue of shape and dimension of the curve conduit 208. Such rigidity allows the curved conduit 208, and by extension, the personal temperature control device 200 to remain in place, and secured to the wearable item, while directing controlled air flow (e.g., cooled air flow or heated air flow) underneath the wearable item. Rigidity of the curved conduit 208 also helps to displace the wearable item outward, allowing controlled air flow provided by the curved conduit 208 to travel beneath the wearable item to provide cooling and/or heating. In some embodiments, a control of air flow, by virtue of controlling the fan 206, for instance, may allow for of a displacement of the wearable item, and hence a cooling and/or heating beneath the wearable item. For example, a higher air flow would result in a higher pressure beneath the wearable item, and hence more displacement of the wearable item.

In some embodiments, a cross-section of the curved conduit 208, and main channel 210 therein, may vary along a path of the main channel 210. For instance, as illustrated in FIGS. 2C and 2F, a cross-section of the curved conduit 208 proximal to a channel inlet of the main channel 210 may be less than the cross-section of the curved conduit 208 proximal to a channel outlet 268. Also, a configuration of the main channel 210 in the curved conduit 208 may also vary to control a direction of air flow, for instance, underneath a wearable item. For instance, as shown in FIG. 2G, in some embodiments, the curved conduit 208 may include a number of ribs 282 positioned proximal to a channel outlet 268 of the main channel 210, where the ribs divide the main channel 210 to form a number of secondary channels 284, each secondary channel 284 directing air flow in a slightly different direction, underneath the wearable item (e.g., FIG. 2C). In this manner, more efficient or effective cooling or heating underneath the wearable item may be achieved. In other words, air flow through secondary channels 284 allow for a larger cooling or heating area beneath the wearable item.

In some embodiments, the curved conduit 208 may also include a conduit ring 286 that extends from the curved conduit 208, and interlocks with a pair of body rings 288 extending from the body of the housing 202. For instance, the conduit ring 286 and the pair of body rings 288 may be secured together using a pin 290 (e.g., FIGS. 2E-2E). Such configuration may reinforce attachment of the curved conduit 208 to the housing 202. In some embodiments, the curved conduit 208 may extend from the housing 202 via a collar 292 (e.g., FIG. 2A). The collar 292 may also reinforce attachment of the curved conduit 208 to the housing 202. In some embodiments, the curved conduit 208 may be directly attached or joined to the housing 202. In such embodiments, direct attachment may be reinforced by using thicker materials, for instance, about or near to the bottom surface 250. As appreciated from FIGS. 2A-2H, reinforcement of the curved conduit 208 may be particularly advantageous since pressure on the curved conduit 208, for instance, upon introducing a wearable item in the space between the curved conduit 208 and the rear surface 256 of the body of the housing 202 may induce substantial strain on the joint or connection between the curved conduit 208 and the housing 202.

As described, the personal temperature control device 200 and/or air flow provided by the personal temperature control device 200 may get warm with time. To this end, the personal temperature control device 200 may implement any number of ways to cool the personal temperature control device 200, and/or components therein, as well as air flowing therethrough. For example, the personal temperature control device 200 may implement a passive cooling approach, for example, via one or more opening, slot, or vent in the housing 202 allowing heat to escape from the body interior 204. For example, as illustrated in FIGS. 2B, 2D-2E, and 2G, the personal temperature control device 200 may include an array of of vents 294, arranged about the housing 202. While the figures illustrate one possible arrangement of vents 294, other arrangements and number of vents 294 may be possible.

The personal temperature control device 200 may alternatively, or additionally, implement an active cooling approach or an active heating approach. For instance, in some embodiments, the personal temperature control device 200 may include a Peltier cell for controlling a temperature of the personal temperature control device 200, and/or various components therein, as well as air flowing therethrough. For instance, in some embodiments, a first side of the Peltier cell may be arranged to face a body exterior, and a second side of the Peltier cell may be arranged to face the body interior. To provide efficient temperature control, in some embodiments, a heat sink may be attached to the first side of the Peltier cell. A conductive mass may also be attached to the second side of the Peltier cell. In some embodiments, at least a portion of air flow provided by the fan 206 may be exposed to the conductive mass (or component thermally linked thereto), thereby controlling a temperature of air flow introduced into the main channel 210. For instance, at least a portion of a flow conduit connecting the output of the fan 206 and the channel inlet of the main channel 210 may be thermally linked to the conductive mass. In some embodiments, a voltage may be applied to the Peltier cell to generate heat on the first side of the Peltier cell, or the second side of the Peltier cell. In other embodiments, the personal temperature control device 200 may include a heating element (e.g., resistive element, and so forth), directly, or indirectly linked (e.g., via a conductive mass) to at least a portion of a flow conduit connecting the output of the fan 206 and the channel inlet of the main channel 210.

As described, the personal temperature control device 200 may include a power source that may store and/or deliver electric power to various components on the personal temperature control device 200, such as the fan 206, Peltier cell, heating element, and so forth. As an example, the power source may include one or more rechargeable battery, one or more removable battery, one or more solar cell, and so forth. Power provided by the power source may be delivered using various components and hardware (e.g., circuitry, circuit board, wiring, and so forth), and may be selectively controlled using various components and hardware (e.g., button, switch, potentiometer, slider, microcontroller, and so forth) arranged in or about the personal temperature control device 200. For example, the personal temperature control device 200 may include a button 296 for operating or activating the fan 206.

As described, the personal temperature control device 200 may also include other components and features for additional use, convenience, and/or functionality of the personal temperature control device 200. For instance, in some embodiments, the personal temperature control device 100 may include a source of light 298, which may be selectively powered by the power source. For example, the source of light 298 may include one or more LED, light bulb, and so forth.

While FIGS. 2A-2H provide one example of a personal temperature control device 200, it may be appreciated that a number of variations may be possible. For instance, while the housing 202 of the personal temperature control device 200 is shown to be substantially rectangular, the housing 202 may have any shape and dimension. For example, the housing 202 may be rectangular, square, oval, round, and so forth. Further, while the curved conduit 208 is illustrated to be substantially rectangular, the curved conduit 208, and main channel 210 therein, may also have any shape and dimension. For example, a cross-section of the curved conduit 208, and/or main channel 210 therein, may have a cross-section that is rectangular, square, oval, round, and so forth.

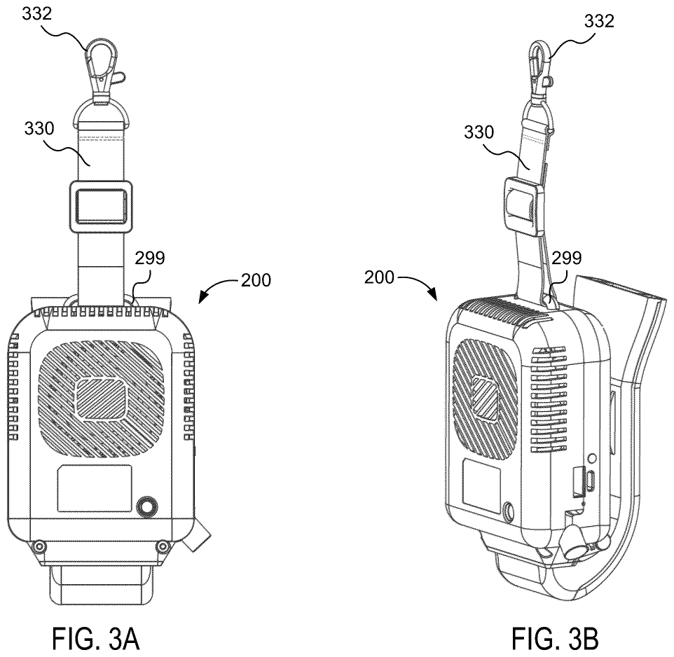

Also, in some embodiments, the body of the personal temperature control device 200 may include a receptacle 299 for attaching a lanyard, strap, and so forth. In some uses, as illustrated in the example of FIGS. 3A-3B, an adjustable lanyard 330 may be attached to the receptacle 299, allowing a user to conveniently carry the personal temperature control device 200. In other uses, the adjustable lanyard 330 may prevent loss or damage or loss to the personal temperature control device 200 by including a fastening device 332 (e.g., fastener, hook, latch, clip, carabiner, and so forth), which may be removably attached to a wearable item. For instance, as shown in FIGS. 3A-3B, the fastening device 332 may include a spring-loaded carabiner. In some embodiments, the adjustable lanyard may include an elastic lanyard.

As appreciated from examples illustrated in FIGS. 2A-2H and 3A-3B, the personal temperature control device 200 provides enhanced functionality compared to conventional temperature control device. For example, the personal temperature control device 200 may be securely attached at a top of the protective vest (thereby providing downward air flow underneath the protective vest), as well as at a bottom of the protective vest (thereby providing upward air flow underneath the protective vest).

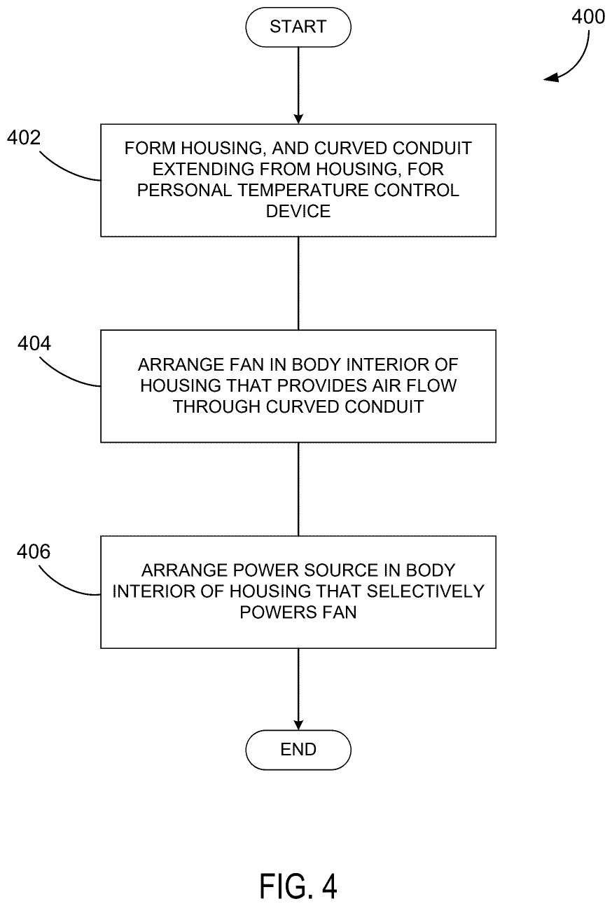

Turning now to FIG. 4, a flowchart setting forth steps of a process 400, according to aspects of the present disclosure, is illustrated. Steps of the process 400 may be carried out using any combination of suitable devices, tools, hardware, systems, and so forth. In some embodiments, steps of the process 400 may be implemented as instructions stored in non-transitory computer-readable media, as a program, firmware or software, and executed by a general-purpose, programmed or programmable computer, processor or other computing device. In other embodiments, steps of the process 400 may be hardwired in an application-specific computer, processor, dedicated system, or module. Although the process 400 is illustrated and described as a sequence of steps, it is contemplated that the steps may be performed in any order or combination, need not include all illustrated steps, and may include additional steps.

The process may begin at process block 402 with forming various components a personal temperature control device, in accordance with aspects of the present disclosure. In particular, process block 402 may include forming a housing having a body with various surfaces (e.g., a bottom surface, a top surface, a front surface, a rear surface, and other surfaces), and a curved conduit extending from the housing, may be formed at process block 402. The housing and curved conduit may be formed using various manufacturing techniques and materials. For instance, the housing and curved conduit may be manufactured using injection molding, compression molding, extrusion molding, rotational molding, thermoforming, transfer molding, blow molding and so forth. In some implementations, the housing and/or curved conduit may be formed from a durable material, such as a plastic, metal, rubber, and so forth.

In some implementations, process block 402 may include forming at least a portion of the curved conduit to angle relative to a plane defined by the rear surface of the body. Also, in some implementations, process block 402 may include forming a cross-section of the curved conduit proximal to a channel inlet to be less than the cross-section of the curved conduit proximal to a channel outlet. Further, in some implementations, process block 402 may including forming the curved conduit with a plurality of ribs positioned proximal to a channel outlet, wherein the plurality of ribs form a number of secondary channels, each directing air flow in a different direction.

In some implementations, process block 402 may include forming the curved conduit with a spring clip slot that may receive a spring clip. As described, the curved conduit and the spring clip may engage a wearable item to secure the personal temperature control device to the wearable item, and direct air flow underneath the wearable item. To this end, process block 402 may include forming and/or attaching the spring clip to the housing. As described, the spring clip may be formed to provide a tension sufficient to hold a weight of the personal temperature control device when the curved conduit and the spring clip engage the wearable item. In some implementations, process block 402 may include forming or arranging the spring clip to extend along an axis defined by the top surface and the bottom surface of the body, where the spring clip is formed with a first curvature and a second curvature along the axis. Forming the first curvature of the spring clip may facilitate an insertion of a portion of the wearable item between the spring clip and the curved conduit, and forming the second curvature of the spring clip may facilitate securing the portion of the wearable item between the spring clip and the curved conduit. In some implementations, the spring clip may be formed with sufficient rigidity to provide a force that may push back against the wearable item and hold a weight of the personal temperature control device when the curved conduit and the spring clip engage the wearable item. The spring clip may also be formed with sufficient elasticity to flex, as well as revert to its original shape when the curved conduit and the spring clip disengage the wearable item.

In some implementations, process block 402 may include forming the body interior with at least one compartment, for instance, to hold the fan, the power source, and so forth. Process block 402 may also include forming a conduit ring that extends from the curved conduit and interlocks with a pair of body rings extending from the body, and securing the conduit ring and the pair of body rings together using a pin. Process block 402 may further include forming the body with a receptacle for attaching, for example, a lanyard or strap. Process block 402 may further include forming the body with at least one opening, a grill, a mesh, or a slot for supplying air to an input of the fan.

While process block 402 above has been described to include various forming steps, it may be appreciated that fewer or more steps may be included. For instance, some or all features described above may be formed simultaneously in one manufacturing step or action, or using one manufacturing technique.

The process 400 may continue with installing or arranging various components in the housing of the personal temperature control device described above. For instance, as indicated by process block 404, a fan may be arranged in the body interior of the housing, where the fan provides controlled air flow through the curved conduit, as described. A power source may also be arranged in the body interior of the housing, which can selectively power the fan, for instance, as indicated by process block 404, as well as other components of the personal temperature control device. Process blocks 404 and 406 may be carried out sequentially, in any order, or simultaneously. Various other components may also be arranged in the body interior of the housing. For instance, in some implementations, a Peltier cell may be arranged therein, which can control a temperature in the body interior, as well as the temperature of air flowing through the personal temperature control device. Further, a number of assembly steps may be carried out at various points along the process 400, including making electrical connections, adjusting/gluing/securing components, and so forth.

While various examples of the present disclosure have been described above, it should be understood that they have been presented by way of example only, and not limitation. Numerous changes to the disclosed examples can be made in accordance with the disclosure herein without departing from the spirit or scope of the disclosure. Thus, the breadth and scope of the present disclosure should not be limited by any of the above-described examples. Rather, the scope of the disclosure should be defined in accordance with the following claims and their equivalents.

Although the disclosure has been illustrated and described with respect to one or more implementations, equivalent alterations and modifications will occur to others skilled in the art upon the reading and understanding of this specification and the annexed drawings. In addition, while a particular feature of the disclosure may have been disclosed with respect to only one of several implementations, such feature may be combined with one or more other features of the other implementations as may be desired and advantageous for any given or particular application.

The terminology used herein is for the purpose of describing particular examples only and is not intended to be limiting of the disclosure. As used herein, the singular forms “a,” “an,” and “the” are intended to include the plural forms as well, unless the context clearly indicates otherwise. Furthermore, to the extent that the terms “including,” “includes,” “having,” “has,” “with,” or variants thereof, are used in either the detailed description and/or the claims, such terms are intended to be inclusive in a manner similar to the term “comprising.”

Unless otherwise defined, all terms (including technical and scientific terms) used herein have the same meaning as commonly understood by one of ordinary skill in the art to which this disclosure belongs. Furthermore, terms, such as those defined in commonly used dictionaries, should be interpreted as having a meaning that is consistent with their meaning in the context of the relevant art, and will not be interpreted in an idealized or overly formal sense unless expressly so defined herein.

What is claimed is:

1. A personal temperature control device comprising:
a housing having a body with a bottom surface, a top surface, a front surface, and a rear surface defining a body interior;
a fan that provides air flow, wherein the fan is arranged in the body interior;
a power source that selectively powers the fan; and
a curved conduit that extends from an opening in the bottom surface and along the rear surface of the body, the curved conduit comprising a main channel connected to an output of the fan,
wherein the curved conduit is configured to direct air flow from the fan and secure the personal temperature control device to a wearable item on a person.

2. The personal temperature control device of claim 1, wherein the curved conduit is rigid.

3. The personal temperature control device of claim 1, wherein the wearable item is a protective vest.

4. The personal temperature control device of claim 1, wherein at least a portion of the curved conduit is angled relative to a plane defined by the rear surface of the body.

5. The personal temperature control device of claim 1, wherein a cross-section of the curved conduit proximal to a channel inlet is less than the cross-section of the curved conduit proximal to a channel outlet.

6. The personal temperature control device of claim 1, wherein the curved conduit further comprises a plurality of ribs positioned proximal to a channel outlet, the plurality of ribs forming a number of secondary channels, each directing air flow in a different direction.

7. The personal temperature control device of claim 1 further comprising a spring clip attached to the rear surface of the body.

8. The personal temperature control device of claim 7, wherein the spring clip is configured to provide a force against the wearable item sufficient to hold a weight of the personal temperature control device when the curved conduit and the spring clip engage the wearable item, and revert to an original shape when the curved conduit and the spring clip disengage the wearable item.

9. The personal temperature control device of claim 7, wherein the curved conduit and the spring clip engage the wearable item to secure the personal temperature control device to the wearable item, and direct air flow underneath the wearable item.

10. The personal temperature control device of claim 7, wherein the spring clip comprises a metallic shim or strip.

11. The personal temperature control device of claim 7, wherein the curved conduit further comprises a spring clip slot that receives the spring clip.

12. The personal temperature control device of claim 7, wherein the spring clip extends along an axis defined by the top surface and the bottom surface of the body, the spring clip comprising a first curvature and a second curvature along the axis.

13. The personal temperature control device of claim 12, wherein the first curvature of the spring clip facilitates an insertion of a portion of the wearable item between the spring clip and the curved conduit, and the second curvature of the spring clip secures the portion of the wearable item between the spring clip and the curved conduit.

14. The personal temperature control device of claim 1, wherein the power source comprises at least one rechargeable battery, at least one removable battery, or at least one solar cell.

15. The personal temperature control device of claim 1, wherein the body interior comprises at least one compartment that holds the fan.

16. The personal temperature control device of claim 15, wherein the at least one compartment further holds the power source for powering the fan.

17. The personal temperature control device of claim 1, wherein the personal temperature control device further comprises a conduit ring that extends from the curved conduit and interlocks with a pair of body rings extending from the body, wherein the conduit ring and the pair of body rings are secured together using a pin.

18. The personal temperature control device of claim 1, wherein the body further comprises a receptacle for attaching a lanyard.

19. The personal temperature control device of claim 1, wherein the body further comprises at least one opening, a grill, a mesh, or a slot for supplying air to an input of the fan.

20. The personal temperature control device of claim 1, wherein the personal temperature control device further comprises a Peltier cell for controlling a temperature of the personal temperature control device.

21. The personal temperature control device of claim 20, wherein a first side of the Peltier cell is arranged to face a body exterior, and a second side of the Peltier cell is arranged to face the body interior.

22. The personal temperature control device of claim 21, wherein a heat sink is attached to the first side of the Peltier cell.

23. The personal temperature control device of claim 22, wherein a conductive mass is attached to the second side of the Peltier cell, wherein at least a portion of the air flow is exposed to the conductive mass to provide controlled air flow to the main channel.

24. A personal temperature control device comprising:
a housing having a body with a bottom surface, a top surface, a front surface, and a rear surface defining a body interior;
a fan that provides air flow, wherein the fan is arranged in the body interior;
a curved conduit that extends from an opening in the bottom surface and along the rear surface of the body, the curved conduit comprising a main channel connected to an output of the fan; and
a spring clip attached to the rear surface of the body,
wherein the curved conduit is configured to engage with the spring clip to secure the personal temperature control device to a wearable item on a person, and direct air flow under the wearable item.

25. The personal temperature control device of claim 24, wherein the curved conduit is rigid.

26. The personal temperature control device of claim 24, wherein the wearable item is a protective vest.

27. The personal temperature control device of claim 24, wherein at least a portion of the curved conduit is angled relative to a plane defined by the rear surface of the body.

28. The personal temperature control device of claim 24, wherein a cross-section of the curved conduit proximal to a channel inlet is less than the cross-section of the curved conduit proximal to a channel outlet.

29. The personal temperature control device of claim 24, wherein the curved conduit further comprises a plurality of ribs positioned proximal to a channel outlet, the plurality of ribs forming a number of secondary channels, each directing air flow in a different direction.

30. The personal temperature control device of claim 24, wherein a shape and dimension of the spring clip provides a tension sufficient to hold a weight of the personal temperature control device when the curved conduit and the spring clip engage the wearable item.

31. The personal temperature control device of claim 24, wherein the spring clip comprises a metallic shim or strip.

32. The personal temperature control device of claim 24, wherein the curved conduit further comprises a spring clip slot that receives the spring clip.

33. The personal temperature control device of claim 24, wherein the spring clip extends along an axis defined by the top surface and the bottom surface of the body, the spring clip comprising a first curvature and a second curvature along the axis.

34. The personal temperature control device of claim 33, wherein the first curvature of the spring clip facilitates an insertion of a portion of the wearable item between the spring clip and the curved conduit, and the second curvature of the spring clip secures the portion of the wearable item between the spring clip and the curved conduit.

35. The personal temperature control device of claim 24 further comprising a power source comprising at least one rechargeable battery, at least one removable battery, or at least one solar cell.

36. The personal temperature control device of claim 24, wherein the body interior comprises at least one compartment that holds the fan.

37. The personal temperature control device of claim 36, wherein the at least one compartment further holds a power source for powering the fan.

38. The personal temperature control device of claim 24, wherein the personal temperature control device further comprises a conduit ring that extends from the curved conduit and interlocks with a pair of body rings extending from the body, wherein the conduit ring and the pair of body rings are secured together using a pin.

39. The personal temperature control device of claim 24, wherein the body further comprises a receptacle for attaching a lanyard.

40. The personal temperature control device of claim 24, wherein the body further comprises at least one opening, a grill, a mesh, or a slot for supplying air to an input of the fan.

41. The personal temperature control device of claim 24, wherein the personal temperature control device further comprises a Peltier cell for controlling a temperature of the personal temperature control device.

42. The personal temperature control device of claim 41, wherein a first side of the Peltier cell is arranged to face a body exterior, and a second side of the Peltier cell is arranged to face the body interior.

43. The personal temperature control device of claim 42, wherein a heat sink is attached to the first side of the Peltier cell.

44. The personal temperature control device of claim 42, wherein a conductive mass is attached to the second side of the Peltier cell, wherein at least a portion of the air flow is exposed to the conductive mass to provide controlled air flow to the main channel.

45. A method for manufacturing a personal temperature control device, the method comprising:
forming a housing having a body with a bottom surface, a top surface, a front surface, and a rear surface defining a body interior, and a curved conduit extending from the bottom surface along the rear surface of the body;
arranging, in the body interior, a fan that provides air flow to a main channel in the curved conduit; and
arranging, in the body interior, a power source that selectively powers the fan,
wherein the curved conduit is configured to direct air flow from the fan and secure the personal temperature control device to a wearable item on a person.

46. The method of claim 45, wherein the method further comprises forming the curved conduit using a rigid material.

47. The method of claim 45, wherein the wearable item is a protective vest.

48. The method of claim 45, wherein the method further comprises forming at least a portion of the curved conduit to angle relative to a plane defined by the rear surface of the body.

49. The method of claim 45, wherein the method further comprises forming a cross-section of the curved conduit proximal to a channel inlet to be less than the cross-section of the curved conduit proximal to a channel outlet.

50. The method of claim 45, wherein the method further comprises forming the curved conduit with a plurality of ribs positioned proximal to a channel outlet, the plurality of ribs forming a number of secondary channels, each directing air flow in a different direction.

51. The method of claim 45, wherein the method further comprises forming a spring clip to provide a force against the wearable item sufficient to hold a weight of the personal temperature control device when the curved conduit and the spring clip engage the wearable item, and revert to an original shape when the curved conduit and the spring clip disengage the wearable item.

52. The method of claim 51, wherein the method further comprises forming the curved conduit and the spring clip to engage the wearable item to secure the personal temperature control device to the wearable item, and direct air flow underneath the wearable item.

53. The method of claim 51, wherein the method further comprises forming the curved conduit with a spring clip slot that receives the spring clip.

54. The method of claim 51, wherein the method further comprises forming the spring clip to extend along an axis defined by the top surface and the bottom surface of the body, wherein the spring clip is formed with a first curvature and a second curvature along the axis.

55. The method of claim 54, wherein the method further comprises forming the first curvature of the spring clip to facilitate an insertion of a portion of the wearable item between the spring clip and the curved conduit, and forming the second curvature of the spring clip to secure the portion of the wearable item between the spring clip and the curved conduit.

56. The method of claim 45, wherein the method further comprises forming the body interior with at least one compartment to hold the fan, the power source, or both.

57. The method of claim 45, wherein the method further comprises forming a conduit ring that extends from the curved conduit and interlocks with a pair of body rings extending from the body, and securing the conduit ring and the pair of body rings together using a pin.

58. The method of claim 45, wherein the method further comprises forming the body with a receptacle for attaching a lanyard.

59. The method of claim 45, wherein the method further comprises forming the body with at least one opening, a grill, a mesh, or a slot for supplying air to an input of the fan.

60. The method of claim 45, wherein the method further comprises arranging a Peltier cell that controls a temperature in the body interior.
